(19) United States
(12) Patent Application Publication
(10) Pub. No.: US 2004/0023372 A1
(43) Pub. Date: Feb. 5, 2004

(54) Tubular nanostructures

(75) Inventors: Michael L. Klein, Ocean City, NJ (US); Carlos Lopez, Philadelphia, PA (US)

Correspondence Address: SYNNESTVEDT & LECHNER, LLP, 2600 ARAMARK TOWER, 1101 MARKET STREET, PHILADELPHIA, PA, 191072950

(73) Assignee: The Trustees of the University of Pennsylvania, Philadelphia, PA

(21) Appl. No.: 10/446,622

(22) Filed: May 28, 2003

Related U.S. Application Data
(60) Provisional application No. 60/383,883, filed on May 28, 2002.

(51) Int. Cl. C12M 1/34; C12N 1/00

(52) U.S. Cl. 435/317.1; 435/287.1

(57) ABSTRACT

The present invention is directed to a tubular nanostructure for providing a stable nanometer-sized pore across a lipid bilayer membrane having a hydrophobic core region between two hydrophilic surface regions comprising a tubular body having a hydrophobic region flanked by hydrophilic regions, a method for inserting a tubular nanostructure into a lipid bilayer membrane, and a method for providing a stable pore in a lipid bilayer membrane.

### CROSS-REFERENCE TO RELATED APPLICATION

This claims the benefit of and incorporates by reference provisional Application No. 60/383,883, filed May 28, 2002.

### FIELD OF THE INVENTION

The present invention is directed to tubular nanostructures for providing a stable, nanometer-sized pore across a membrane. The invention is directed also to a method for inserting a tubular nanostructure into a lipid bilayer membrane and a method for providing a nanometer-sized pore across such a membrane.

### BACKGROUND OF THE INVENTION

Cellular membranes perform several critical functions such as providing a boundary between the cell and the outside world and regulating the transport of materials across that boundary. Within the cell also there are membranes which compartmentalize and help carry out important cellular processes and events. While cellular membranes have diverse functions in the different regions and organelles of a cell, they share a common structure. Cellular membranes are generally comprised of lipid molecules which are characterized as having a polar, hydrophilic head and a hydrophobic tail. The primary lipids of cellular membranes are phospholipids, such as phosphoglycerides, in which glycerol forms the backbone of the molecule and two non-polar hydrocarbon chains of fatty acid residues are present as esters bonded to the glycerol. The third site links to a bridging phosphate group. The other end of the phosphate bridge links to another organic subunit, most commonly a nitrogen-containing alcohol. The phosphate ester portion of the molecule comprises the polar, hydrophilic head. Cellular membranes also may comprise other molecules such as carbohydrates and proteins, which serve a variety of functions such as carrying molecules and providing the membrane with distinctive functional properties.

The lipid molecules comprising a cellular membrane are arranged in two opposing layers to form a membrane structure having a core region and two surface regions. Each layer of lipid molecules, also called a leaflet, consists of a two-dimensional array of lipid molecules having a common orientation in which all of the lipid heads are directed to one side of the layer and all of the lipid tails are directed to the other side of the layer. The two lipid layers that comprise a cellular membrane are arranged such that the lipid heads in each layer face away from each other becoming the two surfaces of the membrane, and the lipid tails in each layer face toward each other becoming the membrane core. In this arrangement, the lipid layers form a bilayer membrane having a hydrophilic exterior and a hydrophobic core region and serve as effective barriers to polar molecules and ions, as well as to large molecules and molecular complexes. This is advantageous to the extent that polar molecules, ions, and various larger substances constitute cell poisons. Yet such substances may also be cell nutrients or therapeutic agents. Thus, it is desirable to have the ability to overcome the barrier presented by a cell membrane in order to transport materials across the membrane.

There are a variety of mechanisms by which substances are transported across a lipid bilayer. Certain materials, such as water, oxygen, carbon dioxide, ethanol and urea, readily cross cell membranes by simple diffusion, and the relative rate of diffusion is roughly proportional to the concentration gradient across the membrane. Many other materials, however, such as, for example, small hydrophilic molecules such as glucose, macromolecules such as proteins and RNA, and ions such as K⁺, Na⁺, Ca²⁺, Cl⁻and HCO³⁻, diffuse poorly across a lipid bilayer membrane either because of their size and/or their charge which causes them to be repelled from like charges in the cell membrane. Their charge can further cause them to electrically bind water molecules, and in hydrated form, become effectively quite large.

There are two transport mechanisms which involve proteins or assemblies of proteins embedded in the cell membrane by which ions and other difficult to diffuse materials travel across a membrane. The carrier transport mechanism involves a membrane-soluble ionophore that encapsulates the material, carries it across the lipid bilayer, and releases it on the other side. Such ionophores are often specific to the type of ion or molecule that it can transport, and may contain a gate to control permeability across the membrane. The channel transport mechanism occurs through a structured pore that spans the lipid bilayer, allowing for flow of the material across the membrane. The structured pore can also be limited in the type of ion or molecule that it can transport as it is comprised of the specific proteins resident on the membrane.

In addition to the aforementioned transport systems, it is known that a new pathway through the cell membrane can be made by means of self-assembling peptide ring nanostructures that can spontaneously insert into the cell membrane. These peptide ring nanostructures comprise cyclic peptides made of an even number of alternating D- and L-amino acids in an open, flat conformation, with all the side chains pointing outward. One example of such a cyclic peptide consists of a ring of 10 amino acids comprising five each of D-leucine and L-tryptophan in alternating arrangement These rings are assembled into a stack by intermolecular hydrogen bonding through the amide backbone thereby forming a nanotube. The properties of the tube surface and inner pore can be varied by modifying the amino acid sequence, and the size is determined by the number of residues. These tubular structures have been used to selectively transport glutamic acid across an artificial cell membrane.

Peptide nanotubes, however, can be problematic in providing stable pore formation for the transport of various materials across a membrane. Small variations in chemical structure can have profound effects on selectivity. For example, one amino acid can be the difference between a sequence that acts only on bacterial cells and another that acts on both bacterial and mammalian cells. Also, peptide nanotubes assemble only at the site of action, and there is no single way that molecules of a cyclic peptide will stack. Rather, the manner of stacking will depend on the conditions the cyclic peptides encounter in the membrane.

Tubular nanostructures, such as carbon nanotubes, have been the subject of much interest for their potential benefits in many small-scale applications. Carbon nanotubes such as single wall carbon nanotubes (SWNTs) are long, thin cylinders of carbon that are unique for their shape, size, and physical properties. A carbon nanotube can be described as a graphene sheet seamlessly wrapped into the form of a cylinder. Carbon nanotubes may be formed in a number of ways including catalytic chemical vapor deposition by carbon arc and laser vaporization techniques in which carbon nanotubes are initiated by the formation of a first closed end, growth along the leading edge at a constant diameter, and then closure at the second end. As a structure substantially comprising an array of carbon atoms, carbon nanotubes are non-polar and, therefore, intrinsically hydrophobic. As described in more detail below, while carbon nanotubes have been considered in forming passageways in lipid bilayer membranes, their purely hydrophobic character renders such nanotubes unsuitable for the formation of a stable membrane pore. For example, when a carbon nanotube is positioned in a lipid bilayer membrane, hydrophobic lipid tails tend to aggregate at and occlude the hydrophobic open ends of the nanotube. As a result, a stable pore is not readily maintained across the membrane.

### SUMMARY OF THE INVENTION

According to one aspect of the present invention, there is provided a tubular nanostructure for providing a stable, nanometer-sized pore across a lipid bilayer membrane comprising a tubular body having a hydrophobic region flanked by hydrophilic regions. Preferably, the hydrophilic regions of the nanostructure comprise functional groups located at the ends of the tubular body. By having a hydrophobic region flanked by hydrophilic regions, the configuration of the tubular nanostructure is compatible with the arrangement of hydrophobic and hydrophilic regions present in a lipid bilayer membrane. When acting as a membrane pore, the hydrophobic region of the tubular nanostructure is positioned across the core of the membrane which is comprised of lipid tails, and the hydrophilic regions of the nanostructure are in proximity to the polar, hydrophilic lipid heads on each surface of the membrane. As a result of this coordination of hydrophobic and hydrophilic regions of the nanostructure and the membrane, the nanostructure is well-suited to maintaining a stable, substantially perpendicular orientation with respect to the plane of a membrane. Moreover, with the assistance of lipid molecules, the nanostructure is also capable of spontaneous penetration and orientation of the nanostructure through the membrane. Positioned across a lipid bilayer membrane, the nanostructure provides for a stable, nanometer-sized pore across the lipid bilayer for transport or conductance activity across the membrane that avoids lipid occlusion associated with hydrophobic nanotubes. The nanostructure of the present invention is particularly well-suited for use in the administration of therapeutic agents or genetic material across a cell membrane.

According to another aspect of the present invention, there is provided a method for inserting a tubular nanostructure into a lipid bilayer membrane. The method comprises the steps of applying to a lipid bilayer membrane the tubular nanostructure of the present invention and allowing the nanostructure to penetrate the membrane spontaneously with the assistance of lipids from the membrane. Preferably, the hydrophilic regions of the nanostructure comprise functional groups located at the ends of the tubular body. The lipid molecules which assist the nanostructure in crossing the membrane core, referred to herein as escort lipids, undergo trans-leaflet lipid flips in which lipids travel from the layer on which the nanostructure is first applied to the other lipid layer of the membrane. The method may be used to establish a stable, nanometer-sized pore in a lipid bilayer membrane for the administration of therapeutic agents or genetic material across the membrane.

According to a further aspect of the present invention, there is provided a method for providing a stable, nanometer-sized pore in a lipid bilayer membrane. The method comprises the step of positioning across a lipid bilayer membrane the tubular nanostructure of the present invention in an orientation substantially perpendicular to the plane of the membrane. The hydrophobic region of the tubular body allows a stable pore to be established across the membrane core while the hydrophilic flanking regions minimizes occlusions of the pore by lipid tails. Preferably, the hydrophobic and hydrophilic regions of the tubular nanostructure are sized to be substantially coincident with the hydrophobic and hydrophilic regions of the membrane.

### BRIEF DESCRIPTION OF THE DRAWINGS

FIG. 1a shows a molecular dynamics simulation of the process by which a coarse-grain model of the tubular nanostructure of the present invention penetrates a membrane.

FIG. 2A shows a stick and ball depiction of a prior art hydrophobic nanotube.

FIG. 2B shows an embodiment of the tubular nanostructure of the present invention in which the hydrophilic regions comprise functional groups located at the ends of the nanostructure.

FIG. 2C shows a schematic representation of the all-atom dimyristoylphosphatidylcholine (DMPC) molecule.

FIG. 2D depicts a coarse-grain model of a DMPC molecule.

FIG. 3A shows Voronoi tessellations for the centers of mass of the lipids and the tubular nanostructure as the nanostructure begins to penetrate the model membrane.

FIG. 3B shows a graphic depiction of the centers of mass of the lipids and the tubular nanostructure as the nanostructure begins to penetrate the model membrane.

FIG. 4 shows a comparison of oscillations over time of a hydrophobic nanotube of the prior art with a tubular nanostructure of the present invention.

### DETAILED DESCRIPTION

The tubular nanostructure of the present invention comprises a tubular body having a hydrophobic region flanked by hydrophilic regions. While either or both of the hydrophilic regions may be located at the ends or inward from the ends of the tubular body, it is preferred that at least one of the hydrophilic regions is located at an end of the tubular body, and more preferably, that both hydrophilic regions are located at the ends of the tubular body. As a structure for forming a pore in a lipid bilayer membrane, and more preferably a cellular membrane, the tubular body of the present invention is preferably sized in its diameter and length with reference to the membrane to be penetrated.

The tubular body may be of any length suitable for the establishment of a passageway across the entire thickness of the membrane. Preferably, the length is selected with reference to the thickness of the hydrophobic and hydrophilic regions of the membrane to be penetrated so that the hydrophobic and hydrophilic regions of the tubular body may be substantially coincident with the hydrophobic and hydrophilic regions of the membrane when positioned therethrough. In preferred form, the tubular nanostructure has a length of about 20 Å to about 40 Å, and more preferably about 30 Å to about 35 Å.

The tubular body may be of any diameter suitable for the establishment of a stable passageway across a membrane. Preferably, the diameter is selected with reference to the membrane to be penetrated and the materials that will be transported across the membrane. And in contrast to the self-assembling peptide nanotubes, the tubular nanostructures of the present invention are assembled away from the site of action which allows for the precise selection of diameter and the application to the membrane of only those nanostructures having the desired pore size for the delivery or evacuation of materials across the membrane. In applications involving the transport of hydrophilic units of a size comparable to a solvated ion with its first solvation shell, the tubular nanostructure preferably has a diameter of about 5 Å to about 20 Å and more preferably about 10 Å to about 15 Å.

The tubular nanostructure may comprise any tubular body that is capable of accommodating a hydrophobic region and hydrophilic regions on either side of the hydrophobic region, preferably at the ends of the tubular body. The tubular body must also be suitable in its dimensions, composition and physical properties for the formation of a pore across a lipid bilayer membrane, preferably a cellular membrane. Preferably, the tubular nanostructure comprises at least one protein, antimicrobial peptide, cyclic peptide, amino acid, graphene sheet, or a natural or synthetic polymer. The nanostructure may be formed directly into tubular shape or from constituent units comprising rings, helices and/or sheets More preferably the tubular nanostructure comprises a carbon nanotube, a crown ether tube, or a natural or synthetic polymer tube. The tubular nanostructure may be provided as a complete, free-standing structure or used as a part of a combination of elements. When part of such a combination, the additional elements may include structures to guide and position the nanostructure in a membrane, such as a bearing, stanchion, boom and the like, and components associated with the delivery or extraction of materials through the nanostructure such as a pump, plunger, meter, reservoir, and the like.

An important aspect of the tubular nanostructure of the present invention is its amphiphilicity and the arrangement of hydrophobic and hydrophilic regions along the tubular body. The nanostructure is provided with both a hydrophobic region and hydrophilic regions located on either side of the hydrophobic region, preferably at or near each end of the tubular body. The hydrophobic region of the tubular nanostructure is designed to be compatible with the hydrophobic lipid tails that occupy the interior region of a lipid bilayer membrane. The hydrophilic regions of the tubular nanostructure are designed to be compatible with the hydrophilic lipid heads comprising the surfaces of a lipid bilayer membrane. In this configuration, the nanostructure is able to be stably oriented across a lipid bilayer membrane substantially perpendicular to the plane of the membrane. The hydrophilic ends of the nanostructure also assist in maintaining the accessibility of the passageway by reducing the incidence of lipid tail occlusion. Further, the hydrophilic regions in combination with lipid molecules, allow for the spontaneous transport of the nanostructure across the membrane core.

The hydrophilic character of the flanking regions of the tubular body may be a result of the intrinsic properties of the tubular body, or as a result of functional groups placed at the ends of the tubular body that exhibit hydrophilic character. Suitable functional groups include any hydrophilic group that is capable of being secured on the tubular body and of imparting a hydrophilic character thereto. The hydrophilic regions of the tubular body may bear the same or different functional groups. In applications in which the membrane is a cellular membrane, it is preferred that the hydrophilic group is also biocompatible. In preferred form, the hydrophilic groups on the nanostructure comprise chemical moieties that are capable of interacting favorably with hydrophilic lipid head groups such as, for example, amines, amides, charged or polar amino acids, alcohols, carboxylic groups, ester groups, ether groups, ester-ether groups, and derivatives thereof. In functionalizing the tubular body to accommodate hydrophilic moieties, the method by which the functional group is added will depend upon, among other things, the particular nanostructure and functional groups that are used.

Another aspect of the present invention is a method for the insertion of a tubular nanostructure into a lipid bilayer membrane to establish a stable passageway therethrough. The method comprises the steps of applying to a lipid bilayer membrane the tubular nanostructure of the present invention and allowing the nanostructure to penetrate the membrane spontaneously with the assistance of lipids from the membrane. Preferably, the hydrophilic regions of the nanostructure comprise functional groups located at the ends of the tubular body. The tubular body is sized to a length and diameter suitable for the establishment of a passageway across a membrane, preferably a cellular membrane.

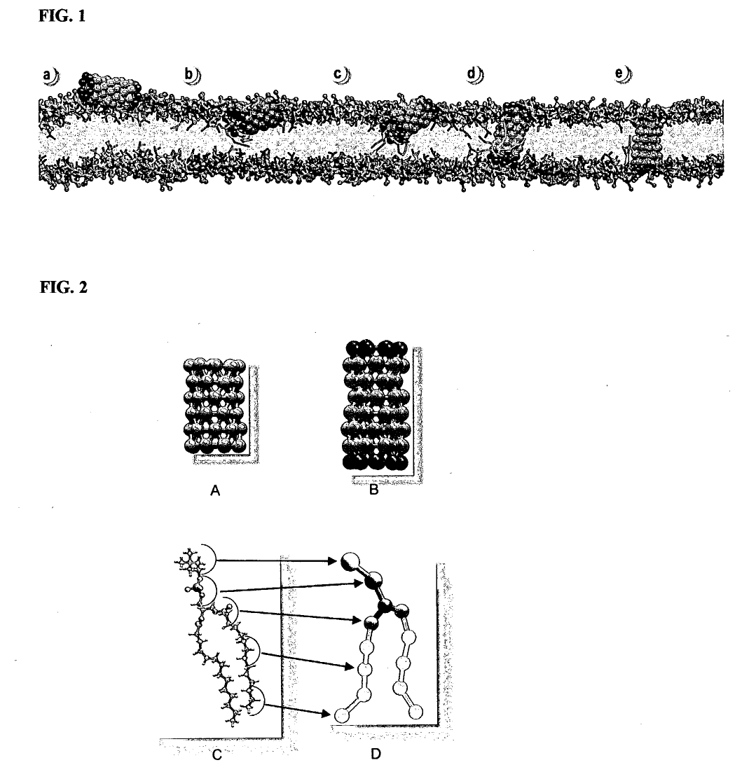

The application step involves the placing of the tubular nanostructure in proximity to the one surface of the membrane. For cellular membranes, the relevant surface is the exterior surface of the cell. As shown in FIG. 1, pore formation in a lipid bilayer membrane occurs in a process whereby the nanostructure of the present invention is first adsorbed onto (FIG. 1 a) and partially immersed in (FIG. 1b) a membrane surface with the long axis of the tubular body parallel to the lipid-water interface. During this process, lipids form salt bridges with the hydrophilic regions of the nanostructure.

In the penetration step of the method, random thermal fluctuations drive one end of the nanostructure towards the core of the membrane as shown in FIG. 1c. The tubular body is assisted in crossing the membrane core by lipid molecules from the membrane whose head groups form salt bridges to the tube caps. Lipids from the first layer contacted by the tubular body remain attached to the immersed hydrophilic region of the nanostructure. These lipids escort the nanostructure across the membrane and occlude the nanostructure end thereby preventing lipid tails from entering the interior of the nanostructure. After this structure passes through the core of the lipid bilayer, the escort lipids and the hydrophilic region interact with the opposite side of the membrane as shown in FIG. 1d. The long axis of the tubular body aligns in a substantially perpendicular orientation to the plane of the membrane. The escort lipids detach from the hydrophilic region once reorientation is complete and become incorporated into the far membrane leaflet thereby undergoing trans-leaflet lipid flips. By analogy, it is believed that positively charged residues in antibiotic molecules adhere to negatively charged lipids, which subsequently escort the transmembrane insertion process and may also play a role in peptide oligomerization. The assistance of the escort lipids allows the tubular nanostructure to form a stable, nanometer-sized pore as shown in FIG. 1e. The pore thus established is capable of transporting water, aqueous solutions, pharmaceuticals, cellular, genetic or other materials across the membrane.

Molecular dynamics (MD) calculations were used to study the interaction of tubular nanostructures with a fully hydrated dimyristoylphosphatidylcholine (DMPC) lipid bilayer in the liquid-crystalline phase. The calculations were performed using a coarse-grain (CG) model and are representative of tubular nanostructures comprising membrane-spanning proteins, antimicrobial peptides, cyclic peptides, or carbon nanotubes. The MD simulations demonstrate that a tubular nanostructure comprising a tubular body having a hydrophobic region flanked by hydrophilic regions spontaneously inserts into, aligns with, and conducts molecules across a lipid bilayer membrane.

Coarse-grain models for nanostructures and lipid molecules are shown in FIG. 2. FIG. 2A depicts a stick-and-ball representation of a prior art hydrophobic nanotube. As shown, the nanotube is hydrophobic along its entire length. FIG. 2B shows a tubular nanostructure of the present invention which comprises a hydrophobic central region similar to the nanotube of the prior art that is flanked at each end by hydrophilic caps. FIG. 2C shows a schematic representation of the all-atom DMPC molecule, and FIG. 2D depicts its coarse grain counterpart in which groups of individual atoms are represented by larger unitary structures.

In a separate molecular dynamics simulation, the nanostructure of the present invention initially perpendicular to the bilayer plane rotates to become parallel to the membrane plane prior to its immersion, and then follows the same insertion pattern as shown in FIG. 1. By comparison, molecular dynamics simulations starting with the purely hydrophobic nanotube of the prior art placed in water results in a slow insertion process and a tube that becomes solvated and occluded by membrane lipid tails.

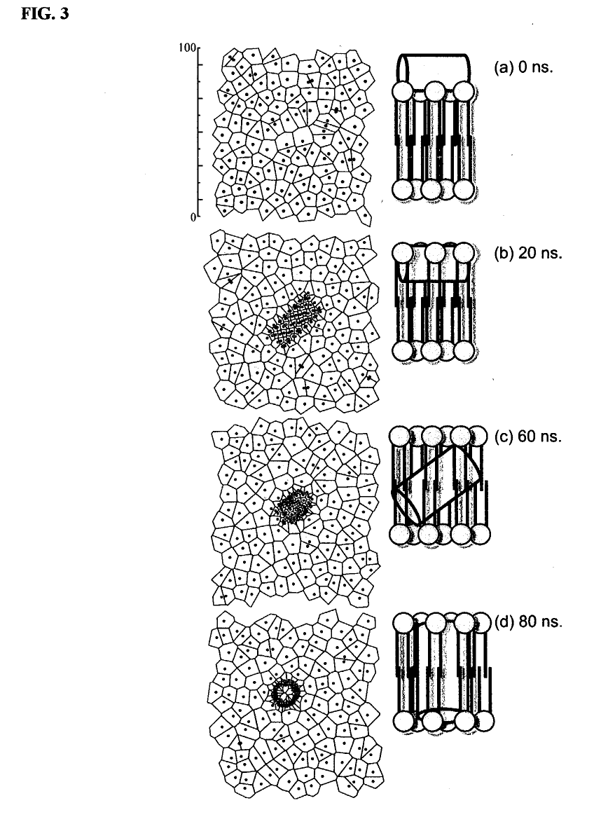

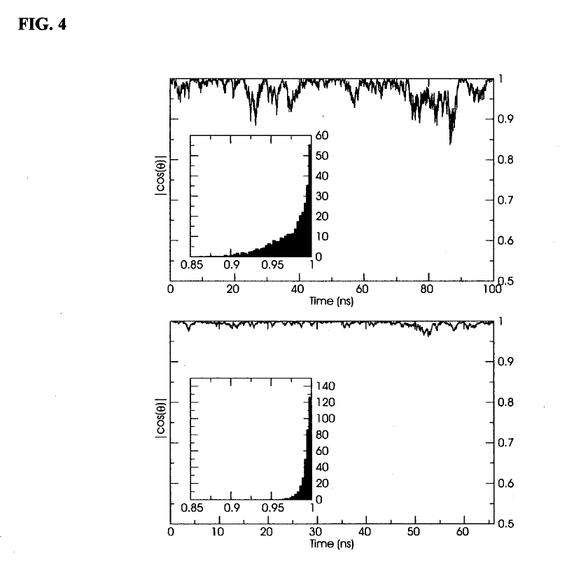

It is believed that there is a direct relationship between the surface tension of a lipid layer and the area per head group of its constituent lipids. This relationship is typically expressed in terms of surface pressure/area per lipid isotherms. Two-dimensional tessellations with Voronoi polyhedra are used to examine local instantaneous changes in the area per head group of the lipids in the vicinity of a nanostructure of the present invention. FIG. 3 shows Voronoi tessellations for the centers of mass of each lipid and the nanostructure sites, and corresponding graphic renditions, as the tubular body penetrates the membrane. At 0 ns as shown in FIG. 3a, the tubular body is adsorbed onto the surface but penetration has not started. At 20 ns as shown in FIG. 3b, the tubular body has penetrated laterally onto the bilayer, and there are two shells of different area per head group surrounding the tubular body. At 60 ns as shown in FIG. 3c, the tubular body is halfway through its rotation, and the area of the lipid atoms in contact with the tubular body changes slowly. At 80 ns as shown in FIG. 3d, the tubular body has become completely inserted into the membrane. The tessellations depicted in FIG. 3 are shown with reference to an Angstrom scale.

During the insertion process, the area per head group of the coarse-grain lipids in the neighborhood of the nanostructure decreases from its equilibrium value of around 70 Å² to about 49 Å² during immersion (FIG. 3b), about 54 Å² after the tubular body has submerged into the membrane (FIG. 3c), about 60 Å² as it rotates, and finally about 56 Å² upon completion of the rotation (FIG. 3d). The lipids in the second shell follow a similar trend in their area per head group changes (within about 5 Å² of their equilibrium value). Thus, only the lipids in the first shell around the tubular body display a significant response to the stress induced by immersion. The lower area per head group value of those lipids immediately surrounding the tubular body is attributed, at least in part, to strongly attractive membrane-nanostructure interactions.

To further characterize the interaction of a tubular nanostructure with a cellular membrane, MD simulations were performed with prior art hydrophobic carbon nanotubes and functionalized carbon nanotubes in accordance with the present invention both initially embedded in a DMPC bilayer and each with their long axes perpendicular to the plane of the membrane surface. Water is observed to populate the pore during the initial stages of the simulation, but subsequently the prior art nanotube tilts and lipid tails enter the ends from both leaflets thereby blocking the transport of water. The occlusion the nanotube is alleviated by the functionalized nanotube of the present invention. In this case, the tilting of the tube is reduced by the presence of hydrophilic ends that can make salt bridges to lipids.

As shown in FIG. 4, the long axes of both the prior art nanotube and the functionalized tubular nanostructure of the present invention remain approximately perpendicular to the membrane plane throughout the simulation, but the tilting oscillations of the prior art nanotube are large enough for lipid tails to enter and occlude the pore. The oscillations in the positioning of the nanotube as shown in FIG. 4A allow for lipid tails to penetrate the lumen of the tube and occlude the pore. Simulations with longer purely hydrophobic tubes resulted in tubes with larger tilt angles and pore occlusion. The oscillations are reduced by the addition of hydrophilic groups to the ends of the nanotube as shown in FIG. 4B. This functionalized nanotube can conduct water sites across the membrane. The embedded graphs represent normalized histograms of the tilt angles collected from the simulations.

As discussed above, it is important to select a nanostructure comprising a hydrophobic region and flanking hydrophilic regions of comparable size to the hydrophobic and hydrophilic subdomains of the membrane in order to achieve a stable pore across the membrane. Further, having the hydrophilic regions located at either end of the tubular body enhances the accessibility of the passageway running through the tubular body and, as mentioned above, allows for the spontaneous transport of the tubular body across the membrane core. Although the functionalized nanotube conducts water throughout the coarse-grain simulation without occlusion by the lipids, such events are too scarce for meaningful statistics to be acquired about this process. However, since the coarse-grain water units used represent three loosely packed water molecules, a fully atomistic simulation of the present system should accommodate a continuous water column or at least a wire across the pore as has been shown. The diameter of the pore is about 13 Å which is appropriately sized for water/ion transport and is comparable to the diameter of viral ion pores which have a hydrophobic core.

In the coarse-grain model, each DMPC lipid molecule consists of 13 interaction sites, eight of which are hydrophobic (four for each alkanoyl tail) and five of which are hydrophilic, three for the glycerol moiety and one each for the charged choline and phosphate as shown in FIG. 2. Groups of three water molecules are considered as a single CG unit (W) with no net charge. With suitable parameterization, the CG DMPC and W particles self-assemble spontaneously to form a stable hydrated bilayer, corresponding to the liquid crystalline L_{α}phase, in quantitative agreement with experimental data. The length of the tubular body was selected to match the width of the hydrophobic core of a CG DMPC membrane. The width of 13 Å was chosen to allow passage of W particles. In total the nanotube has 48 hydrophobic interaction sites uniformly distributed on its surface in a triangular lattice i.e., six evenly separated rings with eight sites each as shown in FIG. 1A. The tubular nanostructure of the present invention contains an additional ring of eight hydrophilic units at each end, for an overall length of 30 Å as shown in FIG. 1B. The hydrophobic and hydrophilic sites share the same parameters as those previously determined for the alkanoyl tails of CG DMPC and W, respectively.

All molecular dynamic simulations described herein employ the CM3D program and the NPT ensemble. All the simulations were carried out at 303.15 K and 1 atm. The van der Waals interactions were truncated at 15 Å while the real part of the electrostatic interactions was truncated at 18.7 Å, and a multiple time step integrator scheme was used to optimize the simulations. The molecular species in each simulation were distributed as follows: the simulation of a functionalized nanotube above a lipid bilayer was composed of the tube, 256 CG DMPC lipids, and 4,263 W sites (representing 12,789 water molecules). The simulations of nanotubes and functionalized nanotubes embedded in a bilayer consisted of 256 CG DMPC lipids, 2,192 W sites (representing 6,576 water molecules) and a single prior art nanotube or functionalized nanotube. All other simulations described herein are geometrical variations of these basic simulations. The interaction potential parameters for the CG model were fit iteratively to the results of all-atom MD simulations on corresponding systems. From previous all-atom and CG simulation studies, it is believed that the CG systems evolve several orders of magnitude faster than their all-atom counterparts, thereby providing access to physical phenomena on larger spatial and temporal scales.

We claim:

1. A tubular nanostructure for providing a stable nanometer-sized pore across a lipid bilayer membrane having a hydrophobic core region between two hydrophilic surface regions comprising a tubular body having a hydrophobic region flanked by hydrophilic regions.

2. The tubular nanostructure of claim 1 wherein at least one of the hydrophilic regions is located at an end of the tubular body.

3. The tubular nanostructure of claim 1 wherein both hydrophilic regions are located at the ends of the tubular body.

4. The tubular nanostructure of claim 1 wherein the hydrophobic region of the tubular body is sized to the hydrophobic core region of the membrane and the hydrophilic regions of the tubular body are adjacent to the hydrophobic region of the tubular body

5. The tubular nanostructure of claim 1 wherein tubular body comprises a carbon nanotube and the hydrophilic regions of the tubular body are functional groups selected from the group consisting of amines, amides, charged or polar amino acids, alcohols, carboxylic groups, ester groups, ether groups, ester-ether groups, and derivatives thereof.

6. The tubular nanostructure of claim 1 wherein the tubular body has a length of about 20 Å to about 40 Å.

7. The tubular nanostructure of claim 1 wherein the tubular body has a diameter of about 5 Å to about 20 Å.

8. A method for inserting a tubular nanostructure into a lipid bilayer membrane comprising the steps of applying to a lipid bilayer membrane the tubular nanostructure of claim 1 and allowing the nanostructure to penetrate the membrane spontaneously with the assistance of lipids from the membrane.

9. The method of claim 8, wherein at least one of the hydrophilic regions of the nanostructure is located at an end of the tubular body.

10. The method of claim 8, wherein the tubular body comprises at least one protein, antimicrobial peptide, cyclic peptide, amino acid, graphene sheet, carbon nanotube or a natural or synthetic polymer.

11. The method of claim 8, wherein the hydrophilic regions of the tubular body are functional groups selected from the group consisting of amines, amides, charged or polar amino acids, alcohols, carboxylic groups, ester groups, ether groups, ester-ether groups, and derivatives thereof.

12. The method of claim 8, wherein the tubular body is assisted in crossing the membrane core by lipid molecules from the membrane.

13. The method of claim 12 wherein the lipid molecules assisting the nanostructure in crossing the membrane undergo trans-leaflet lipid flips.

14. A method for providing a stable pore in a lipid bilayer membrane comprising the step of positioning across a lipid bilayer membrane the tubular nanostructure of claim 1.

15. The method of claim 14 wherein at least one of the hydrophilic regions of the tubular nanostructure is located at an end of the tubular body.

16. The method of claim 14 wherein both hydrophilic regions are located at the ends of the tubular body.

17. The method of claim 14 wherein the hydrophobic region of the tubular body is sized to the hydrophobic core region of the membrane and the hydrophilic regions of the tubular body are adjacent to the hydrophobic region of the tubular body

18. The method of claim 14 wherein tubular body comprises a carbon nanotube and the hydrophilic regions of the tubular body are functional groups selected from the group consisting of amines, amides, charged or polar amino acids, alcohols, carboxylic groups, ester groups, ether groups, ester-ether groups, and derivatives thereof.

19. The method of claim 14 wherein tubular body is substantially perpendicular to the membrane.
